(19) United States
(12) Patent Application Publication
(10) Pub. No.: US 2025/0223617 A1
(43) Pub. Date: Jul. 10, 2025

(54) METHOD FOR SYNTHESIZING POLY(3-HYDROXYBUTYRATE) FROM CRUDE GLYCEROL BY USING ESCHERICHIA COLI

(71) Applicant: AVATACK CO., LTD., New Taipei City (TW)

(72) Inventors: Doan-Thanh TA, New Taipei City (TW); Yun-Peng CHAO, Taichung City (TW); Chung-Jen CHIANG, Taichung City (TW); Chu Hsuan HUANG, Taichung City (TW); Chih-Kai CHANG, New Taipei City (TW)

(73) Assignee: AVATACK CO., LTD., New Taipei City (TW)

(21) Appl. No.: 18/409,253

(22) Filed: Jan. 10, 2024

(51) Int. Cl. C12P 7/625; C12N 1/20; C12R 1/19

(52) U.S. Cl. CPC C12P 7/625; C12N 1/205; C12R 2001/19

(57) ABSTRACT

The present invention provides a method for synthesizing poly(3-hydroxybutyrate) from crude glycerol by using Escherichia coli, which includes a glycerol assimilation step and a strain genetic modification step. The glycerol assimilation step is to pass the crude glycerol through the extracellular membrane of Escherichia coli, so that the crude glycerol can enter the cells of Escherichia coli through uptake of a glycerol facilitator and is transformed into dihydroxyacetone phosphate through anaerobic and aerobic pathways. The strain genetic modification step includes a PHB-8 strain synthetic pathway, so that a λP_{L} promoter is constructed to control a phaCAB genome, and then a PHB-8 strain producing poly(3-hydroxybutyrate) is produced. Thereby, the metabolic pathway of Escherichia coli is reprogramed to convert the crude glycerol into acetyl-CoA and NADPH, and in turn increase the yield of poly(3-hydroxybutyrate).

### FIELD OF THE INVENTION

The present invention relates to a method for synthesizing poly(3-hydroxybutyrate) from crude glycerol, in particular to a method for synthesizing acetyl-CoA and NADPH by reprogramming the metabolic pathway of Escherichia coli.

### BACKGROUND OF THE INVENTION

In today's life, plastic is a common material. The plastic can be divided into thermoplastic plastic or thermosetting plastic depending on whether it is deformed after heating. Among them, the thermoplastic plastic includes polyethylene, polypropylene, polystyrene and polyvinyl chloride, and its composition will not change after being heated, so that the thermoplastic plastic can be heated repeatedly, while the thermosetting plastic can only be melted and molded once, which makes the thermosetting plastic unable to be re-heated and molded. However, both polyethylene and polypropylene are products formed through processing petroleum. Moreover, petrochemical products such as polyethylene and polypropylene will exist in the living environment, resulting in a threat to ecology and human health.

However, polyhydroxyalkanoate is regarded as biodegradable plastic, which is an intracellular polyester synthesized by bacteria and has the characteristics of biodegradability, biocompatibility and optical activity, so that the polyhydroxyalkanoate can be applied to the fields of biodegradable packaging materials, tissue engineering materials, sustained-release materials, electrical materials, medical materials and the like, wherein in the family of polyhydroxyalkanoates, poly-3-hydroxybutyrate has attracted the attention of the industry because of its mechanical properties similar to polypropylene. However, the poly(3-hydroxybutyrate) cannot be widely used due to its too high production cost.

### SUMMARY OF THE INVENTION

An objective of the present invention is to reprogram the metabolic pathway of Escherichia coli in a process of synthesizing poly(3-hydroxybutyrate), so that crude glycerol is converted to synthesize acetyl-CoA and NADPH, thereby increasing the yield of the poly(3-hydroxybutyrate).

In order to achieve the aforementioned objective, the present invention relates to a method for synthesizing poly(3-hydroxybutyrate) from crude glycerol by using Escherichia coli, which includes the following steps: a glycerol assimilation step and a strain genetic modification step. The glycerol assimilation step is to pass crude glycerol through an extracellular membrane of Escherichia coli, so that the crude glycerol can enter the cells of the Escherichia coli through uptake of a glycerol facilitator, and is transformed into dihydroxyacetone phosphate through an anaerobic pathway and an aerobic pathway; and the strain genetic modification step includes a PHB-8 strain synthetic pathway, which uses a pHK-Pha plasmid to integrate and insert a phaCAB genome controlled by a λP_{L} promoter into an HK att site of a PHB-6 strain, thereby generating a PHB-8 strain of poly(3-hydroxybutyrate).

The PHB-6 strain has a trc promoter, a λP_{R} promoter, the λP_{L} promoter, a Φ80::λP_{L}-phaCAB, a pta gene, a λ::λP_{L}-phaCAB, a gltA-lacO, a Ptrc-pntAB and a λP_{L}-aceEF. The trc promoter can control a glpF gene. The λP_{R} promoter can control a gldA gene, a dhaK gene and a zwf gene. The λP_{L} promoter can control a pgl gene. The PHB-8 strain has the trc promoter, the λP_{R} promoter, the λP_{L} promoter, the Φ80::λP_{L}-phaCAB, the pta gene, the λ::λP_{L}-phaCAB, the gltA-lacO, the Ptrc-pntAB, the λP_{L}-aceEF and an HK::λP_{L}-phaCAB. The trc promoter can control the glpF gene. The λP_{R} promoter can control the gldA gene, the dhaK gene and the zwf gene. The λP_{L} promoter can control the pgl gene.

In a preferred embodiment, the method for synthesizing poly(3-hydroxybutyrate) from crude glycerol by using Escherichia coli further has a pre-culture step, which is carried out before the glycerol assimilation step, and the pre-culture step is to put the PHB-6 strain into an Erlenmeyer flask, so that the PHB-6 strain is continuously shaken in the Erlenmeyer flask and cultured at a temperature of 30° C.-40° C. for a time of at least 8 hours.

The genetic modification step further includes a PHB-6 strain synthetic pathway, which is to conduct in situ fusion of the λP_{L} promoter with an aceEF genome of a PHB-5 strain by applying a pBlue-aceE plasmid to generate the PHB-6 strain of the poly(3-hydroxybutyrate).

The in situ fusion is a technology of homologous DNA recombination through a λRed recombination system, which is a recombination system from a λ phage, and the λRed recombination system can engineer the homologous DNA so that a target fragment of the homologous DNA can be integrated and inserted into a chromosome of a prokaryote.

However, the genetic modification step further includes a PHB-5 strain synthetic pathway, which is to fuse the trc promoter with a pyridine nucleotide transhydrogenase of a PHB-4 strain to perform the in situ fusion to produce the PHB-5 strain of the poly(3-hydroxybutyrate), so that the PHB-5 strain has the trc promoter, the λP_{R} promoter, the λP_{L} promoter, the Φ80::λP_{L}-phaCAB, the pta gene, the λ::λP_{L}-phaCAB, the gltA-lacO and the Ptrc-pntAB, wherein the trc promoter can control the glpF gene, the λP_{R} promoter can control the gldA gene, the dhaK gene and the zwf gene, and the λP_{L} promoter can control the pgl gene.

Furthermore, the strain genetic modification step further includes a PHB-4 strain synthetic pathway, which is to obtain a passenger DNA from a pBlue-gltO plasmid, and carry out the in situ fusion of a lacO with a citrate synthase gene of a PHB-3 strain to produce the PHB-4 strain of the poly(3-hydroxybutyrate), so that the PHB-4 strain has the trc promoter, the λP_{R} promoter, the λP_{L} promoter, the Φ80::λP_{L}-phaCAB, the pta gene, the λ::λP_{L}-phaCAB and the gltA-lacO, wherein the trc promoter can control the glpF gene, the λP_{R} promoter can control the gldA gene, the dhaK gene and the zwf gene, and the λP_{L} promoter can control the pgl gene.

However, the strain genetic modification step further includes a PHB-3 strain synthetic pathway, which is to integrate and insert the phaCAB genome controlled by the λP_{L} promoter into a λ att site of a PHB-2 strain by using a pLam-Pha plasmid, so that the PHB-3 strain of the poly(3-hydroxybutyrate) is produced by integrating and inserting a λP_{L}-phaCAB into the PHB-2 strain, and in turn the PHB-3 strain has the trc promoter, the λP_{R} promoter, the λP_{L} promoter, the Φ80::λP_{L}-phaCAB, the pta gene and the λ::λP_{L}-phaCAB, wherein the trc promoter can control the glpF gene, the λP_{R} promoter can control the gldA gene, the dhaK gene and the zwf gene, and the λP_{L} promoter can control the pgl gene.

In a preferred embodiment, the strain genetic modification step further includes a PHB-2 strain synthetic pathway, which uses a P1 phage in the Escherichia coli to remove the pta gene in a PHB-1 strain, thereby producing the PHB-2 strain of the poly(3-hydroxybutyrate), so that the PHB-2 strain has the trc promoter, the λP_{R} promoter, the λP_{L} promoter, the Φ80::λP_{L}-phaCAB and the pta gene, wherein the trc promoter can control the glpF gene, the λP_{R} promoter can control the gldA gene, the dhaK gene and the zwf gene, and the λP_{L} promoter can control the pgl gene.

Finally, the strain genetic modification step further includes a PHB-1 strain synthetic pathway, which is to convert the glucose-6-phosphate into a ribulose-5-phosphate under oxidation by an N31 strain having the trc promoter and the λP_{R} promoter through a pentose phosphate pathway. The trc promoter can control the glpF gene, and the λP_{R} promoter can control the gldA gene and a dhaKLM gene. Under the control of the λP_{L} promoter, the passenger DNA can be amplified in a pPR-zwf plasmid or a pSPL-pgl plasmid by a polymerase chain reaction, and the homologous DNA recombination can be conducted on each passenger DNA through the λRed recombination system via an electroporation action. Moreover, a pPhi80-Pha plasmid is used for integrating and inserting the phaCAB genome controlled by the λP_{L} promoter into a Φ80 att site, thereby allowing the λP_{L}-phaCAB to be integrated and inserted into the N31 strain to produce the PHB-1 strain of the poly(3-hydroxybutyrate), and in turn the PHB-1 strain has the trc promoter, the λP_{R} promoter, the λP_{L} promoter, and the Φ80::λP_{L}-phaCAB, wherein the trc promoter can control the glpF gene, the λP_{R} promoter can control the gldA gene, the dhaK gene and the zwf gene, and the λP_{L} promoter can control the pgl gene.

In the pentose phosphate pathway, a zwf gene and a pgl gene are added into the N31 strain, so that an NADPH of the N31 strain is increased.

The present invention is characterized in that the method for synthesizing poly(3-hydroxybutyrate) includes the pre-culture step, the crude glycerol assimilation step and the strain genetic modification step. The pre-culture step is to put the PHB-6 strain into the Erlenmeyer flask, so that the PHB-6 strain can be continuously shaken in the Erlenmeyer flask and cultured at the temperature of 30° C.-40° C. for a time of at least 8 hours. The crude glycerol assimilation step is to pass the crude glycerol through Escherichia coli and thus pass through the extracellular membrane of Escherichia coli, so that the crude glycerol can enter the cells of Escherichia coli through the uptake of the glycerol facilitator and is transformed into dihydroxyacetone phosphate through anaerobic and aerobic pathways. The strain genetic modification step has the PHB-8 strain synthetic pathway, wherein the PHB-8 strain synthetic pathway is to integrate and insert the phaCAB genome controlled by the λP_{L} promoter into the HK att site of the PHB-6 strain by using the pHK-Pha plasmid, and in turn generate the PHB-8 strain of the poly(3-hydroxybutyrate), so that the PHB-8 strain has the trc promoter, the λP_{R} promoter, the λP_{L} promoter, the Φ80::λP_{L}-phaCAB, the pta gene, the λ::λP_{L}-phaCAB, the gltA-lacO, the Ptrc-pntAB, the λP_{L}-aceEF and the HK::λP_{L}-phaCAB. The trc promoter can control the glpF gene, the λP_{R} promoter can control the gldA gene, the dhaK gene and the zwf gene, the λP_{L} promoter can control the pgl gene, and in turn the crude glycerol can be used by reprogramming the metabolic pathways related to the synthesis of acetyl-CoA and NADPH in Escherichia coli, thereby increasing the yield of the poly(3-hydroxybutyrate).

### BRIEF DESCRIPTION OF THE DRAWINGS

FIG. 1 is a flow chart of a method for synthesizing poly(3-hydroxybutyrate) from crude glycerol by using Escherichia coli;

FIG. 2 is a schematic diagram of the synthesis of the poly(3-hydroxybutyrate) from crude glycerol by using Escherichia coli;

FIG. 3 is a graph of fed-batch fermentation of PHB-8 strain fermented for 12-16 hours; and

FIG. 4 is a genetic characteristics of strains applied.

### DETAILED DESCRIPTION OF THE PREFERRED EMBODIMENTS

In order to further understand the construction, use and characteristics of the present invention definitely, fully and accurately, preferred embodiments are given, which are described in detail hereafter in connection with the accompanying drawings.

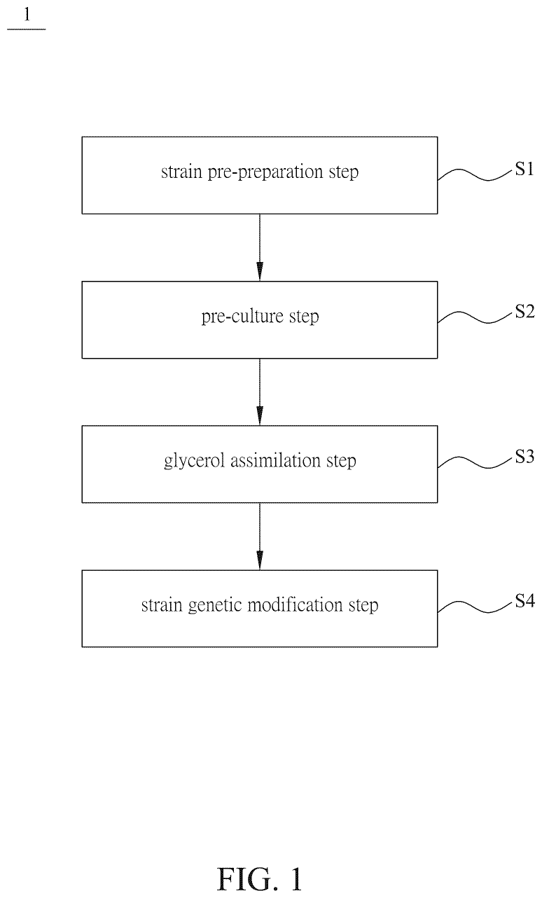

Referring to FIG. 1, a method for synthesizing poly(3-hydroxybutyrate) from crude glycerol by using Escherichia coli in the present invention has a strain pre-preparation step S1, a pre-culture step S2, a glycerol assimilation step S3 and a strain genetic modification step S4.

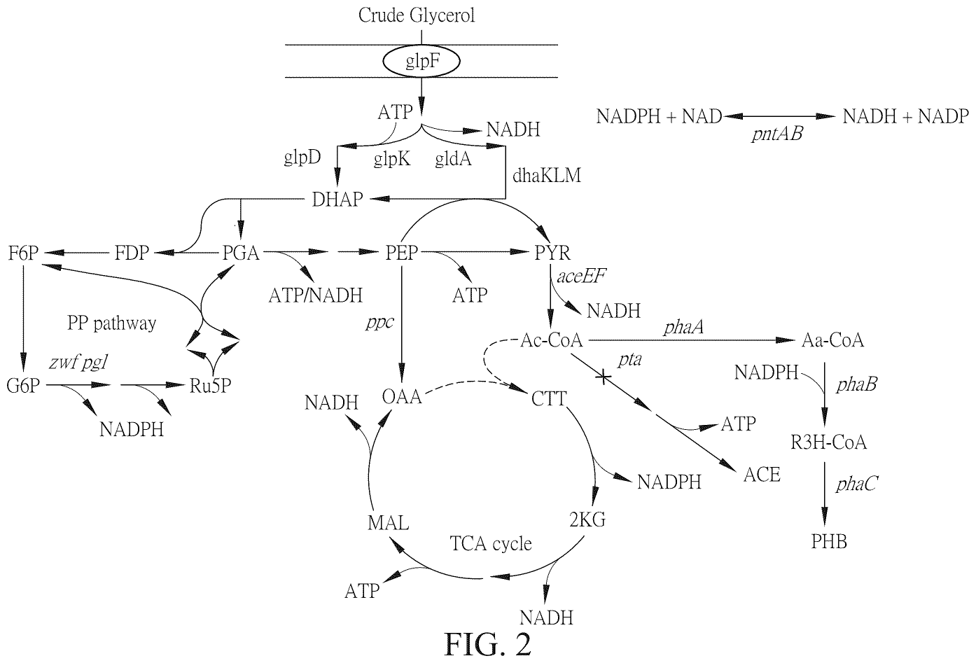

Referring to FIGS. 1 and 2, firstly, the strain pre-preparation step S1 is carried out. Firstly, a Pha1 (gaagaattcagagagacaatcaaatcatgg) primer and a Pha2 (aggcctcgaggtcagcccatatgcaggc) primer are subjected to a polymerase chain reaction to obtain a phaCAB genome of C. necator, and after EcoRI-XhoI enzyme cleavage treatment, a deoxyribonucleic acid subjected to the polymerase chain reaction is incorporated into a pND707 plasmid, thereby producing a pND-Pha plasmid. Subsequently, the polymerase chain reaction is carried out by using the pND-Pha plasmid as a template and using the Pha2 (aggcctcgaggtcagcccatatgcaggc) primer and a Pha3 (ggatggatcctaacaccgtgcgtgtttgac) primer, thereby generating a λP_{L} promoter and the deoxyribonucleic acid that drives a phaCAB genome. Thereafter, at the same time, the deoxyribonucleic acid subjected to the polymerase chain reaction is digested by a BamHI restriction enzyme, and an integrated plasmid based on a phage is digested by the BamHI restriction enzyme and cleaved by an SmaI restriction enzyme, wherein the integrated plasmid includes a pLam-LoxKm plasmid, a pPhi80-LoxKm plasmid and a pHK-LoxKm plasmid, so that the deoxyribonucleic acid subjected to the polymerase chain reaction has a pLam-Pha plasmid, a pPhi80-Pha plasmid and a pHK-Pha plasmid. Moreover, an N31 strain has a trc promoter and a λP_{R} promoter, wherein the trc promoter can control a glpF gene, and the λP_{R} promoter can control a gldA gene and a dhaKLM genome, thereby completing the strain pre-preparation step S1.

Referring to FIGS. 1 and 2, after the strain pre-preparation step S1 is completed, the pre-cultivation step S2 is started. In this embodiment, the pre-cultivation step S2 is to put an initial strain into a 125 mL Erlenmeyer flask with 5 mL of an LB medium so that the initial strain can be soaked in the LB medium; then, the Erlenmeyer flask is placed in a heating stirrer, so that the initial strain in the Erlenmeyer flask can be continuously shaken in the LB medium at a rotating speed of 200 rpm and cultured at a temperature of 30° C.-40° C. for a time of at least 8 hours; thereafter, the shaken initial strain is inoculated into a shake flask with 15 mL of an improved M9 minimal medium. In this embodiment, the improved M9 minimal medium including 20 g/L of crude glycerol and 1 g/L of a yeast extract, is composed of salts, and contains 1 g/L of ammonium chloride, 6.04 g/L of sodium hydrogen phosphate, 3 g/L of potassium dihydrogen phosphate, 0.5 g/L of sodium chloride, 0.24 g/L of magnesium sulfate, 0.022 g/L calcium chloride and a trace mineral. The trace mineral includes 8 mg/L of ferrous sulfate heptahydrate, 1.31 mg/L of aluminum sulfate, 0.2 mg/L of goslarite, 0.1 mg/L of copper chloride dihydrate, 0.2 mg/L of sodium molybdate dihydrate, 0.74 mg/L of manganese chloride, 0.07 mg/L of cobalt chloride hexahydrate and 0.05 mg/L of boric acid. The crude glycerol includes 63.5% (w/w) of glycerol, 1.7% of a fatty acid and 4.6% of ash. Moreover, a light at a wavelength of 550 nm is used for measuring the growth of cells, wherein the culture of the strain is conducted by taking an initial cell density detected by the light at the wavelength of 550 nm as 0.1.

Referring to FIGS. 1 and 2, after the pre-culture step S2 is completed, the glycerol assimilation step S3 is started. In this embodiment, the crude glycerol passes through an extracellular membrane of Escherichia coli from the periphery of the Escherichia coli. The crude glycerol can enter the cells of Escherichia coli through uptake of a glycerol facilitator, so that the crude glycerol can pass through the extracellular membrane from the periphery of the Escherichia coli and thus enter the cells of the Escherichia coli in a diffusion-facilitating manner, wherein the glycerol facilitator belongs to a water-transporting membrane protein for transporting the crude glycerol, thereby making the crude glycerol facilitator be a highly selective channel protein. However, after the crude glycerol enters the cells of Escherichia coli, the crude glycerol is mainly converted into dihydroxyacetone phosphate through two metabolic pathways: an anaerobic pathway and an aerobic pathway. As shown in FIG. 2, in the anaerobic pathway, the crude glycerol enters a gldA gene after leaving from the glycerol facilitator, the gldA gene can convert the crude glycerol into DHA and generate an NADH to provide energy, which is then converted into the dihydroxyacetone phosphate through a dhaKLM gene. However, in the aerobic path, the crude glycerol enters a glpK gene after leaving from the glycerol facilitator, the glpK gene can phosphorylate the crude glycerol into G3P, but this process needs to consume an ATP, and then the G3P is converted into the dihydroxyacetone phosphate through a glpD gene.

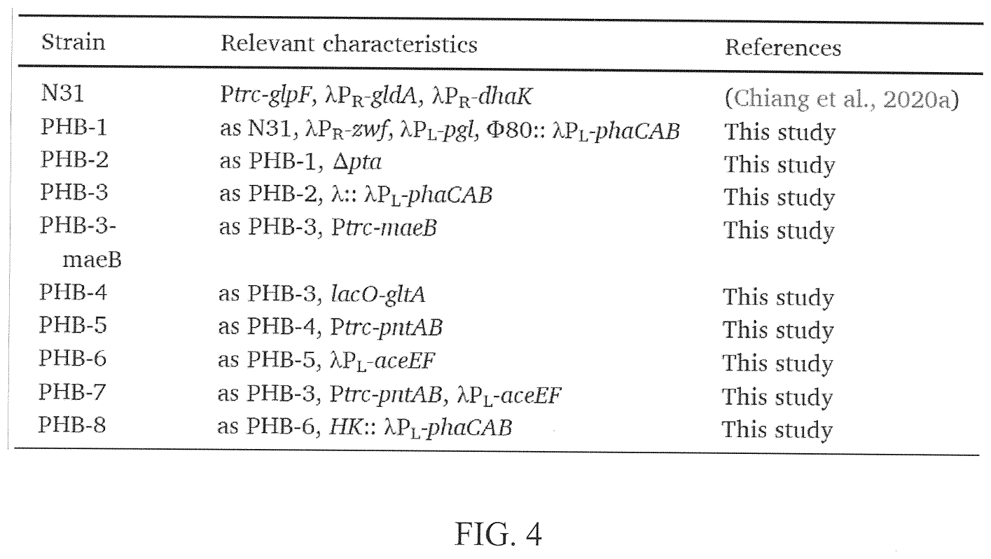

Referring to FIGS. 1, 2 and 4, after the glycerol assimilation step S3 is completed, the strain genetic modification step S4 is started. In this embodiment, the strain genetic modification step S4 includes a PHB-1 strain synthetic pathway, a PHB-2 strain synthetic pathway, a PHB-3 strain synthetic pathway, a PHB-4 strain synthetic pathway, a PHB-5 strain synthetic pathway, a PHB-6 strain synthetic pathway and a PHB-8 strain synthetic pathway, wherein in the PHB-1 strain synthetic pathway, the N31 strain is taken as the initial strain, which converts glucose-6-phosphate into ribulose-5-phosphate through a pentose phosphate pathway, so that the N31 strain is engineered by enhancing the expression of a zwf gene and a pgl gene in the pentose phosphate pathway and adding the zwf gene and the pgl gene into the N31 strain, thereby increasing an NADPH of the N31 strain, and the glucose-6-phosphatase is converted into ribulose-5-phosphate through oxidation. Further, under the control of the λP_{L} promoter, a pPR-zwf plasmid or a pSPL-pgl plasmid is subjected to the polymerase chain reaction with the N31 strain to amplify a passenger DNA, and through an electroporation action, the homologous DNA recombination technology can be conducted on each passenger DNA through a λRed recombination system, so that the zwf gene and the pgl gene in the N31 strain can be fused with the λP_{L} promoter. Moreover, the pPhi80-Pha plasmid is used for integrating and inserting the λP_{L} promoter into a Φ80 att site, and thus integrating and inserting the λP_{L}-phaCAB into the N31 strain to produce the PHB-1 strain of poly(3-hydroxybutyrate), so that the PHB-1 strain has the trc promoter, the λP_{R} promoter, the λP_{L} promoter and a Φ80::λP_{L}-phaCAB, wherein the trc promoter can control the glpF gene, the λP_{R} promoter can control the gldA gene, the dhaK gene and the zwf gene, and the λP_{L} promoter can control the pgl gene.

Referring to FIGS. 1, 2 and 4, the PHB-2 strain synthetic pathway is to take a JW2294-1 (Δpta-779::kan) strain as a donor of a phosphoacetyltransferase gene, and the phosphoacetyltransferase gene catalyzes the conversion into acetophosphate by an acetocoenzyme. Also, a P1 phage in the Escherichia coli is used, through the transduction of the P1 phage as a medium, the phosphoacetyltransferase gene of the PHB-1 strain is deleted to retain the acetyl coenzyme, and the PHB-1 strain is taken as the initial strain, and the acetyl coenzyme is used as a precursor of the synthesis of the poly(3-hydroxybutyrate), thereby producing a PHB-2 strain of the poly(3-hydroxybutyrate). Therefore, the PHB-2 strain has the trc promoter, the λP_{R} promoter, the λP_{L}l promoter, the D80::λP_{L}-phaCAB and a pta gene, wherein the trc promoter can control the glpF gene, the λP_{R} promoter can control the gldA gene, the dhaK gene and the zwf gene, and the λP_{L} promoter can control the pgl gene. However, in the PHB-2 strain synthetic pathway, it will cause that a acetate of the PHB-2 strain decreases sharply, while the yield of the poly(3-hydroxybutyrate) increases by two times, and this acetate is the main by-product of the N31 strain and the poly(3-hydroxybutyrate). Furthermore, after the PHB-2 strain synthetic pathway is ended, significant 2-ketoglutaric acid and pyruvic acid will be produced, so that the usable acetyl coenzyme can be guided into the PHB-3 strain synthetic pathway and a tricarboxylic acid cycle.

Referring to FIGS. 1, 2 and 4, the PHB-3 strain synthetic pathway is to integrate and insert the phaCAB genome controlled by the λP_{L} promoter into a λ att site of a PHB-2 strain by using a pLam-Pha plasmid, and the PHB-2 strain is taken as the initial strain, so that the PHB-3 strain of the poly(3-hydroxybutyrate) is produced by integrating and inserting the λP_{L}-phaCAB into the PHB-2 strain, and in turn the reduced acetyl coenzyme enters into the TCA cycle, and thus the yield of poly(3-hydroxybutyrate) increases and the 2-ketoglutaric acid and pyruvic acid will gradually decrease. Therefore, the PHB-3 strain has the trc promoter, the λP_{R} promoter, the λP_{L} promoter, the 80::λP_{L}-phaCAB, the pta gene and the λ::λP_{L}-phaCAB, wherein the trc promoter can control the glpF gene, the λP_{R} promoter can control the gldA gene, the dhaK gene and the zwf gene, and the λP_{L} promoter can control the pgl gene.

Referring to FIGS. 1, 2 and 4, in this embodiment, that PHB-4 strain synthetic route is to obtain the passenger DNA from a pBlue-gltO plasmid, and carry out the in situ fusion of a lacO with a citrate synthase gene of a PHB-3 strain to remove an endogenous P2 promoter to generate a PHB-4 strain of the poly(3-hydroxybutyrate), and the PHB-3 strain is taken as the initial strain. The activity of the TCA cycle is reduced by limiting the activity of the citrate synthase gene, and thus the acetyl-CoA is retained, so that the PHB-4 strain has the trc promoter, the λP_{R} promoter, the λP_{L} promoter, the Φ80::λP_{L}-phaCAB, the pta gene, the λ::λP_{L}-phaCAB and a gltA-lacO, wherein the trc promoter can control the glpF gene, the λP_{R} promoter can control the gldA gene, the dhaK gene and the zwf gene, and the λP_{L} promoter can control the pgl gene, wherein the in situ fusion is a technology of carrying out homologous DNA recombination through the λRed recombination system. The λRed recombinant system is a recombinant system from a λ phage, and the λRed recombinant system can engineer the homologous DNA so that a target fragment of the homologous DNA can be integrated and inserted onto a chromosome of a prokaryote.

Referring to FIGS. 1, 2 and 4, in this embodiment, the PHB-5 strain synthetic pathway is to fuse a pyridine nucleotide transhydrogenase of the PHB-4 strain with the trc promoter for in situ fusion to produce a PHB-5 strain of the poly(3-hydroxybutyrate) (as shown following).

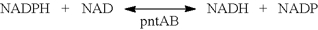

Continually referring to FIGS. 1, 2 and 4, in the PHB-5 strain synthetic pathway, PHB-4 strain is taken as the initial strain, and the NADPH is increased by enhancing the pyridine nucleotide transhydrogenase, so that the PHB-5 strain has the trc promoter, the λP_{R} promoter, the λP_{L} promoter, the Φ80::λP_{L}-phaCAB, the pta gene, the λ::λP_{L}-phaCAB, the gltA-lacO and the Ptrc-pntAB, wherein the trc promoter can control the glpF gene, the λP_{R} promoter can control the gldA gene, the dhaK gene and the zwf gene, and the λP_{L} promoter can control the pgl gene.

Referring to FIGS. 1, 2 and 4, in this embodiment, the PHB-6 strain synthetic pathway is to carry out the in situ fusion of the λP_{L} promoter and an aceEF genome F of the PHB-5 strain by applying a pBlue-aceE plasmid to generate a PHB-6 strain of the poly(3-hydroxybutyrate), and the PHB-5 strain is taken as the initial strain, so that the PHB-6 strain has the trc promoter, the λP_{R} promoter, the λP_{L} promoter, the Φ80::λP_{L}-phaCAB, the pta gene, the λ::λP_{L}-phaCAB, the gltA-lacO, the Ptrc-pntAB and a λP_{L}-aceEF, wherein the trc promoter can control the glpF gene, the λP_{R} promoter can control the gldA gene, the dhaK gene and the zwf gene, and the λP_{L} promoter can control the pgl gene.

Referring to FIGS. 1, 2 and 4, in this embodiment, the PHB-8 strain synthetic pathway is to integrate and insert the phaCAB genome controlled by the λP_{L} promoter into an HK att site of the PHB-6 strain by using the pHK-Pha plasmid, so as to produce a PHB-8 strain of the poly(3-hydroxybutyrate), so that the PHB-8 strain has the trc promoter, the λP_{R} promoter, the λP_{L} promoter, the Φ80::λP_{L}-phaCAB, the pta gene, the λ::λP_{L}-phaCAB, the gltA-lacO, the Ptrc-pntAB, the λP_{L}-aceEF and an HK:λP_{L}-phaCAB promoter, wherein the trc promoter can control the glpF gene, the λP_{R} promoter can control the gldA gene, the dhaK gene and the zwf gene, and the λP_{L} promoter can control the pgl gene.

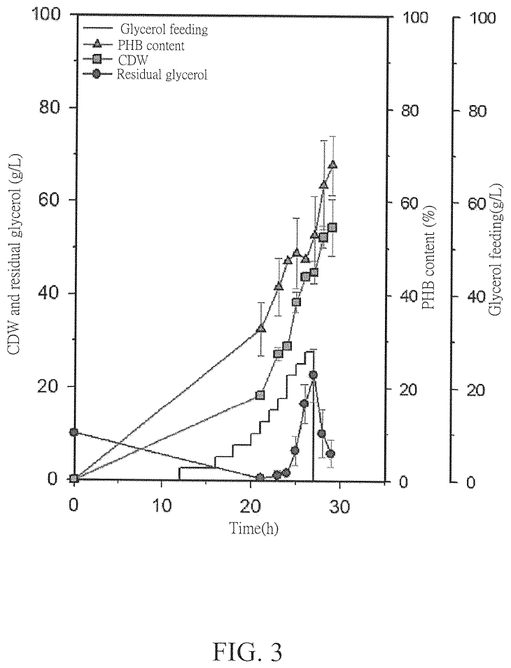

Referring to FIGS. 1, 2 and 3, in this embodiment, after the strain genetic modification step S4 is completed, firstly the PH8 strain is placed in an environment with a temperature of 35-40° C., a dissolved oxygen saturation of 5-15% and a pH value maintained at 5-9 for fermentation for 12-16 hours, wherein the pH value is adjusted by using 25-30% ammonia water (NH₄OH). Then the PHB-8 strain which has been fermented for 12-16 hours is added with a fed-batch solution for fed-batch fermentation. In this embodiment, the fed-batch solution contains salts of an improved M9 minimal medium and 400 g/L of the crude glycerol, so that the PHB-8 strain can be continuously fermented and cultured for 27-31 hours to produce a PHB yield of 25-45 g/L. Therefore, the PHB content in the PHB-8 strain reaches 60-70% and 93-98% of the 400 g/L of the crude glycerol is consumed. Then, a analysis and calculation step of poly(3-hydroxybutyrate) is started. In this embodiment, the PHB-8 strain which has been subjected to the strain genetic modification step S4 is placed in a centrifuge and centrifuged at a rotation speed of 3,000 to 5,000 rpm for 10-20 minutes, and then washed repeatedly with double-distilled water. The double-distilled water containing the PHB-8 strain is collected into and dried in an oven at a temperature of 50-60° C. for at least 8 hours, and then weighed to determine the cell dry weight (CDW) of the PHB-8 strain, so as to obtain the net weight of the PHB-8 strain after moisture removal. The dried PHB-8 strain is collected in a glass tube and treated with concentrated sulfuric acid at a temperature of 80-100° C. for 20-40 minutes, and then the glass tube is cooled at room temperature, and added with 3 mL-5 mL of sulfuric acid (H₂SO₄) at the equivalent concentration of 0.010 N-0.020 N. After vortex flowing in the glass tube, the solution is filtered by a 0.45 μm filter membrane, and the filtered solution is placed in a high performance liquid chromatography (HPLC) instrument for analysis, so as to know the content of crotonic acid, whereby the PHB productivity (g/L/h) can be calculated by lysing the PHB-8 strain and converting the poly(3-hydroxybutyrate) in the PHB-8 strain into the crotonic acid. A PHB yield (g/g) is defined as the PHB yield (g) produced by consuming 1 g of the crude glycerol. Furthermore, the analysis and calculation step of poly(3-hydroxybutyrate) is not limited to only calculating the PHB-8 strain that has been subjected to the strain genetic modification step S4, it can also be used for the PHB-1 strain, the PHB-2 strain, the PHB-3 strain, the PHB-4 strain, the PHB-5 strain, the PHB-6 strain and the PHB-8 strain that have been subjected to the strain genetic modification step S4.

Furthermore, in FIG. 2, the Crude Glycerol represents the crude glycerol, the glpF represents the glycerol facilitator, the gldA represents a glycerol dehydrogenase, the dhaKLM represents a dihydroxyacetone kinase, the DHAP represents the dihydroxyacetone phosphate, the glpK represents a glycerol kinase, the glpD represents a glycerol 3-phosphate dehydrogenase, a PGA represents a 3-phosphoglyceraldehyde, an FDP represents fructose-bisphosphate, the F6P represents the fructose-6-phosphate, the G6P represents the glucose-6-phosphate, the zwf represents a glucose-6-phosphatase dehydrogenase, the pgl represents a lactonase, the Ru5P represents the ribulose-5-phosphate, a PEP represents phosphoenolpyruvate, the PYR represents the pyruvate, the poxB represents the pyruvate oxidase, an ACE represents an acetate, the aceEF represents a pyruvate dehydrogenase (kP_{L}-aceEF), an Aa-CoA represents an acetoacetyl coenzyme A, a phaA represents a β-ketothiolase, a phaB represents an acetoacetyl coenzyme A reductase, a phaC represents a PHB synthase, a R3H-CoA represents (R)3-hydroxybutyryl coenzyme A, the PHB represents the poly(3-hydroxybutyrate), the Ac-CoA represents the acetyl-CoA, the pta represents the phosphotransacetylase, an ACE represents the acetate, a ppc represents a hydroxylase, an OAA represents an oxaloacetate, the gltA represents the citrate synthase gene, a CIT represents a citrate, the 2KG represents the 2-ketoglutaric acid, the MAL represents the malic acid, the maeB represents the malic enzyme, the pntAB represents the pyridine nucleotide transhydrogenase, the TCA cycle represents the tricarboxylic acid cycle, and the PP pathway represents the pentose phosphate pathway.

What is claimed is:

1. A method for synthesizing poly(3-hydroxybutyrate) from crude glycerol by using Escherichia coli, comprising:
a glycerol assimilation step: passing the crude glycerol through an extracellular membrane of the Escherichia coli, so that the crude glycerol can enter the cells of the Escherichia coli through uptake of a glycerol facilitator, and is transformed into dihydroxyacetone phosphate through an anaerobic pathway and an aerobic pathway; and
a strain genetic modification step, comprising: a PHB-8 strain synthetic pathway, which uses a pHK-Pha plasmid to integrate and insert a phaCAB genome controlled by a λP_{L} promoter into an HK att site of a PHB-6 strain, thereby generating a PHB-8 strain of the poly(3-hydroxybutyrate);
wherein the PHB-6 strain has a trc promoter, a λP_{R} promoter, the λP_{L} promoter, a Φ80::λP_{L}-phaCAB, a pta gene, a λ::λP_{L}-phaCAB, a gltA-lacO, a Ptrc-pntAB and a λP_{L}-aceEF; the PHB-8 strain has the trc promoter, the λP_{R} promoter, the λP_{L} promoter, the Φ80::λP_{L}-phaCAB, the pta gene, the λ::λP_{L}-phaCAB, the gltA-lacO, the Ptrc-pntAB, the λP_{L}-aceEF and an HK::λP_{L}-phaCAB promoter, wherein the trc promoter can control a glpF gene, the λP_{R} promoter can control a gldA gene, a dhaK gene and a zwf gene, and the λP_{L} promoter can control a pgl gene.

2. The method for synthesizing poly(3-hydroxybutyrate) from crude glycerol by using Escherichia coli according to claim 1, wherein there is a pre-culture step carried out before the glycerol assimilation step, and the pre-culture step is to put the PHB-6 strain into an Erlenmeyer flask, so that the PHB-6 strain is continuously shaken in the Erlenmeyer flask and cultured at a temperature of 30° C.-40° C. for a time of at least 8 hours.

3. The method for synthesizing poly(3-hydroxybutyrate) from crude glycerol by using Escherichia coli according to claim 1, wherein the genetic modification step further comprises a PHB-6 strain synthetic pathway, which is to conduct in situ fusion of the λP_{L} promoter with an aceEF genome of a PHB-5 strain by applying a pBlue-aceE plasmid to generate the PHB-6 strain of the poly(3-hydroxybutyrate).

4. The method for synthesizing poly(3-hydroxybutyrate) from crude glycerol by using Escherichia coli according to claim 3, wherein the in situ fusion is a technology of homologous DNA recombination through a λRed recombination system, which is a recombination system from a λ phage, and the λRed recombination system can engineer the homologous DNA so that a target fragment of the homologous DNA can be integrated and inserted onto a chromosome of a prokaryote.

5. The method for synthesizing poly(3-hydroxybutyrate) from crude glycerol by using Escherichia coli according to claim 3, wherein the genetic modification step further comprises a PHB-5 strain synthetic pathway, which is to fuse the trc promoter with a pyridine nucleotide transhydrogenase of a PHB-4 strain to perform the in situ fusion to produce the PHB-5 strain of the poly(3-hydroxybutyrate), so that the PHB-5 strain has the trc promoter, the λP_{R} promoter, the λP_{L} promoter, the Φ80::λP_{L}-phaCAB, the pta gene, the λ::λP_{L}-phaCAB, the gltA-lacO and the Ptrc-pntAB, wherein the trc promoter can control the glpF gene, the λP_{R} promoter can control the gldA gene, the dhaK gene and the zwf gene, and the λP_{L} promoter can control the pgl gene.

6. The method for synthesizing poly(3-hydroxybutyrate) from crude glycerol by using Escherichia coli according to claim 5, wherein the strain genetic modification step further comprises a PHB-4 strain synthetic pathway, which is to obtain a passenger DNA from a pBlue-gltO plasmid, and carry out the in situ fusion of a lacO with a citrate synthase gene of a PHB-3 strain to produce the PHB-4 strain of the poly(3-hydroxybutyrate), so that the PHB-4 strain has the trc promoter, the λP_{R} promoter, the λP_{L} promoter, the Φ80::λP_{L}-phaCAB, the pta gene, the λ::λP_{L}-phaCAB and the gltA-lacO, wherein the trc promoter can control the glpF gene, the λP_{R} promoter can control the gldA gene, the dhaK gene and the zwf gene, and the λP_{L} promoter can control the pgl gene.

7. The method for synthesizing poly(3-hydroxybutyrate) from crude glycerol by using Escherichia coli according to claim 6, wherein the strain genetic modification step further comprises a PHB-3 strain synthetic pathway, which is to integrate and insert the phaCAB genome controlled by the λP_{L} promoter into a λ att site of a PHB-2 strain by using a pLam-Pha plasmid, so that the PHB-3 strain of the poly(3-hydroxybutyrate) is produced by integrating and inserting a λP_{L}-phaCAB into the PHB-2 strain, and in turn the PHB-3 strain has the trc promoter, the λP_{R} promoter, the λP_{L} promoter, the Φ80::λP_{L}-phaCAB, the pta gene and the λ::λP_{L}-phaCAB, wherein the trc promoter can control the glpF gene, the λP_{R} promoter can control the gldA gene, the dhaK gene and the zwf gene, and the λP_{L} promoter can control the pgl gene.

8. The method for synthesizing poly(3-hydroxybutyrate) from crude glycerol by using Escherichia coli according to claim 7, wherein the strain genetic modification step further comprises a PHB-2 strain synthetic pathway, which uses a P1 phage in the Escherichia coli to remove the pta gene in a PHB-1 strain, thereby producing the PHB-2 strain of the poly(3-hydroxybutyrate), so that the PHB-2 strain has the trc promoter, the λP_{R} promoter, the λP_{L} promoter, the Φ80::λP_{L}-phaCAB and the pta gene, wherein the trc promoter can control the glpF gene, the λP_{R} promoter can control the gldA gene, the dhaK gene and the zwf gene, and the λP_{L} promoter can control the pgl gene.

9. The method for synthesizing poly(3-hydroxybutyrate) from crude glycerol by using Escherichia coli according to claim 8, wherein the strain genetic modification step further comprises a PHB-1 strain synthetic pathway, which is to convert the glucose-6-phosphate into a ribulose-5-phosphate under oxidation by an N31 strain having the trc promoter and the λP_{R} promoter through a pentose phosphate pathway, the trc promoter can control the glpF gene, and the λP_{R} promoter can control the gldA gene and a dhaKLM gene; under the control of the λP_{L} promoter, the passenger DNA can be amplified in a pPR-zwf plasmid or a pSPL-pgl plasmid by a polymerase chain reaction, and the homologous DNA recombination can be conducted on each passenger DNA through the λRed recombination system via an electroporation action; moreover, a pPhi80-Pha plasmid is used for integrating and inserting the phaCAB genome controlled by the λP_{L} promoter into a Φ80 att site, thereby allowing the λP_{L}-phaCAB to be integrated and inserted into the N31 strain to produce the PHB-1 strain of the poly(3-hydroxybutyrate), and in turn the PHB-1 strain has the trc promoter, the λP_{R} promoter, the λP_{L} promoter, and the Φ80::λP_{L}-phaCAB, wherein the trc promoter can control the glpF gene, the λP_{R} promoter can control the gldA gene, the dhaK gene and the zwf gene, and the λP_{L} promoter can control the pgl gene.

10. The method for synthesizing poly(3-hydroxybutyrate) from crude glycerol by using Escherichia coli according to claim 9, wherein in the pentose phosphate pathway, a zwf gene and a pgl gene are added into the N31 strain, so that an NADPH of the N31 strain is increased.
